(19) United States
(12) Patent Application Publication
(10) Pub. No.: US 2005/0288359 A1
(43) Pub. Date: Dec. 29, 2005

(54) Agents and methods for the treatment of disorders associated with oxidative stress

(75) Inventors: Surindar Singh Cheema, Donvale (AU); Steven Langford, Narre Warren North (AU); Nam Sang Cheung, Singapore (SG); Philip Mark Beart, Ivanhoe (AU); Katherine Jane Macfarlane, Huntingdale (AU); Mark Mulcair, Wantirna South (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: Monash University, Clayton (AU)

(21) Appl. No.: 10/515,722

(22) PCT Filed: May 27, 2003
(86) PCT No.: PCT/AU03/00644

§ 371 Date: Aug. 1, 2005

(30) Foreign Application Priority Data
May 27, 2002 (AU) PS 2554

(51) Int. Cl. A61K 31/335; A61K 31/24

(52) U.S. Cl. 514/450; 514/534

(57) ABSTRACT

The invention provides a method for preventing or reducing the effects of oxidative stress on a substrate. The method includes the step of treating the substrate with a compound of formula (I), or a pharmaceutically acceptable salt thereof, wherein: R¹ is one or more substituents selected from —H, -alkyl, -alkoxy, -aryl, -aryloxy, -halogen, -amino (mono-, di- and tri-substituted), -alkylthio, —NO₂, —COOH, —COOAlkyl, —CO-alkyl, —CN; R² is one or more substituents selected from —H, -alkyl, —(CH₂CH₂O)n-R⁵, a sugar moiety; R³ is —H, -alkyl, -aryl, -alkylOR⁶, -alkylC(O)R⁶; R⁵ is selected from —H, -alkyl, -aryl; and R⁴ and R⁶ are independently selected from —OH, —O-alkyl, —O-polyalkyleneoxy, —O-aryl, —OC(O)O-alkyl, —S-alkyl and -amino.

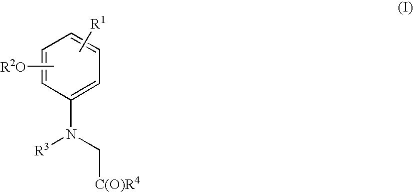

### FIELD OF THE INVENTION

The present invention relates to methods for treating disorders that are associated with oxidative stress such as neurodegenerative disorders. The invention also relates to new chemical entities for use in the treatment of disorders associated with oxidative stress, and more particularly to bis(o aminophenoxy)ethane-N,N,N′,N′-tetraacetic acid (BAPTA) analogues and their use in the treatment of neurodegenerative disorders.

### BACKGROUND OF THE INVENTION

Free radicals are extremely reactive chemical species that cause significant destruction in biological systems. Indiscriminate reaction of free radicals with biological molecules can lead to the destruction of cells and cellular components (e.g. mitochondria), thereby affecting physiological processes by causing cells to lose their structure and/or function.

In biological systems, free radicals are generally referred to as ‘reactive oxygen species’ (ROS). ROS are derived from endogenous sources via the metabolism of oxygen containing species, and from exogenous sources such as toxins and atmospheric pollutants.

Attack of ROS on biological molecules is referred to as ‘oxidative stress’. Oxidative stress has been implicated as a causative factor in a number of degenerative diseases associated with aging, such as Parkinson's disease, Alzheimer's disease, Motor Neuron Disease as well as to Huntington's Chorea, diabetes and Friedreich's Ataxia, and to non-specific damage that accumulates with aging. It also contributes to inflammation and ischemic-reperfusion tissue injury in stroke and heart attack, and also during organ transplantation and surgery.

Oxidative stress occurs when there is an excess of ROS, a decrease in antioxidant levels, or both. Accordingly, agents that interfere with the production of ROS or eliminate ROS may be used to treat many of the disorders associated with neurodegeneration (neurodegenerative disorders), such as Alzheimer's and Parkinson's disease. For example, free radical scavengers (FRS), such as vitamin E, have been shown to reduce neurodegeneration and prolong the life of transgenic mice that develop motor neuron disease.

Throughout this specification reference may be made to documents for the purpose of describing various aspects of the invention. However, no admission is made that any reference cited in this specification constitutes prior art. In particular, it will be understood that the reference to any document herein does not constitute an admission that any of these documents forms part of the common general knowledge in the art in Australia or in any other country. The discussion of the references states what their authors assert, and the applicant reserves the right to challenge the accuracy and pertinency of any of the documents cited herein.

### SUMMARY OF THE INVENTION

The present invention arises out of the inventor's discovery of a new class of antioxidant compounds. These compounds may be used in applications in which it is desirable to prevent or decrease the formation of ROS. These applications include treatment of animals or plants to prevent or cure disorders that result from oxidative stress. Treatment of neurodegenerative disorders in humans and other animals is exemplary of one such application of these compounds. Also, ROS mediated cell damage is implicated in aging and therefore the antioxidant properties of the compounds of the present invention may be utilised as anti-aging agents in cosmetics. Further, there may be other industries (such as the chemical industry) where it is desirable to prevent oxidation of a substrate, and therefore the antioxidants of the present invention may be used in applications related to those industries.

Accordingly, the present invention provides a method for preventing or reducing the effects of oxidative stress on a substrate, the method including the step of treating the substrate with a compound of formula (I), or a pharmaceutically acceptable salt thereof: 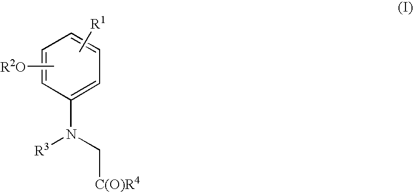
wherein:
R¹ is one or more substituents selected from —H, -alkyl, -alkoxy, -aryl, -aryloxy, -halogen, -amino (mono-, di- and tri-substituted), -alkylthio, —NO₂, —COOH, —COOAlkyl, —CO-alkyl, —CN;
R² is one or more substituents selected from —H, -alkyl, —(CH₂CH₂O)ₙ—R⁵, a sugar moiety;
R³ is —H, -alkyl, -aryl, -alkylOR⁶, -alkylC(O)R⁶;
R⁵ is selected from —H, -alkyl, -aryl; and
R⁴ and R⁶ are independently selected from —OH, —O-alkyl, —O-polyalkyleneoxy, —O-aryl, —OC(O)O-alkyl, —S-alkyl and -amino.

Preferably, R⁵ has the following formula: 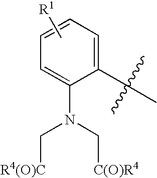
wherein:
R¹ is one or more substituents selected from —H, -alkyl, -alkoxy, -aryl, -aryloxy, -halogen, -amino, -alkylthio, —NO₂, —COOH, —COOAlkyl, —CO-alkyl, —CN; and
R⁴ is selected from —OH, —O-alkyl, —O-polyalkyleneoxy, —O-aryl, —OC(O)O-alkyl, —S-alkyl and -amino.

The present invention also provides a method for preventing or reducing the effects of oxidative stress on a biological system, the method including the step of treating the biological system with a compound of formula (I), or a pharmaceutically acceptable salt thereof: 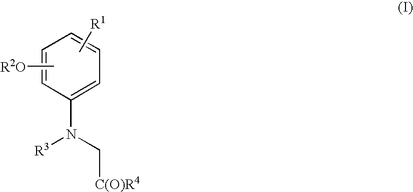
wherein:
R¹ is one or more substituents selected from —H, -alkyl, -alkoxy, -aryl, -aryloxy, -halogen, -amino (mono-, di- and tri-substituted), -alkylthio, —NO₂, —COOH, —COOAlkyl, —CO-alkyl, —CN;
R² is one or more substituents selected from —H, -alkyl, —(CH₂CH₂O)ₙ—R⁵, a sugar moiety;
R³ is —H, -alkyl, -aryl, -alkylOR⁶, -alkylC(O)R⁶;
R⁵ is selected from —H, -alkyl, -aryl; and
R⁴ and R⁶ are independently selected from —OH, —O-alkyl, —O-polyalkyleneoxy, —O-aryl, —OC(O)O-alkyl, —S-alkyl and -amino.

Preferably, R⁵ has the following formula: 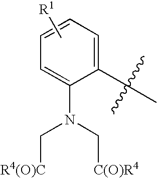
wherein:
R¹ is one or more substituents selected from —H, -alkyl, -alkoxy, -aryl, -aryloxy, -halogen, -amino, -alkylthio, —NO₂, —COOH, —COOAlkyl, —CO-alkyl, —CN; and
R⁴ is selected from —H, —O-alkyl, —O-polyalkyleneoxy, —O-aryl, —OC(O)O-alkyl, —S-alkyl and -amino.

The methods of the present invention may be used to treat disorders or conditions associated with oxidative stress in humans or animals. In one specific form, the method may be used to prevent or cure neurodegenerative disorders. Optionally, the treatment of neurodegenerative disorders may involve administration of an antioxidant compound according to the present invention, in conjunction with another agent for treating a neurodegenerative disorder (e.g. Riluzole, antisense DNA or its analogues such as peptide nucleic acids, neurotrophic factors such as leukaemia inhibitory factor, neurotrophins (NGF, BDNF, NT-3, NT 4/5), glial derived neurotrophic factor (GDNF), lipoic acid or nicotine derivatives).

Antioxidant compounds of formula (I) may also be used in the preparation of a medicament for the treatment of disease states associated with oxidative stress.

The precise triggers and molecular cascades that drive neurodegenerative processes associated with ischaemia, injury and neurodegenerative disorders are poorly understood. However, the present inventors propose that a number of neurological disorders are initiated via dysregulation of Ca²⁺ homeostasis as well oxidative stress pathways. Accordingly, the present invention also provides a method for treating a disease state that is associated with calcium toxicity and oxidative stress, the method including the step of administering a therapeutically effective amount of a free radical scavenger and a calcium buffer.

Preferably, the disease state that is associated with calcium toxicity and oxidative stress is a neurodegenerative disorder such as stroke, epilepsy, Parkinson's disease, Huntington's disease, amyotrophic lateral sclerosis, multiple sclerosis, ageing, ischemia and Alzheimer's disease.

The method may involve administration of a first agent that is free radical scavenger and a second agent that is a calcium buffer. Suitable free radical scavengers include lipoic acid, 2,3-dihydro-1-benzofuran-5-ols, chromanones, trolox, butylated hydroxyl toluene (BHT) and vitamin E. Suitable calcium buffers include derivatives of 15-crown-5,18-crown-6, ethylenediamine tetraacetic acid (EDTA), ethyleneglycol tetraacetic acid (EGTA), cyclohexane-1,2-diamine tetraacetic acid (CDTA) and bis(oaminophenoxy)ethane-N,N,N′,N′-tetraacetic acid (BAPTA). However, the method preferably involves administration of a single agent that is both a free radical scavenger and a calcium buffer. Most preferably the single agent is a compound of formula (I).

Derivatives of bis(oaminophenoxy)ethane-N,N,N′,N′-tetraacetic acid (BAPTA) according to formula (I) have been prepared by the present inventors and these derivatives have been shown to be free radical scavengers and calcium buffers. Accordingly, the present invention also provides a compound of formula (II), or a pharmaceutically acceptable salt thereof: 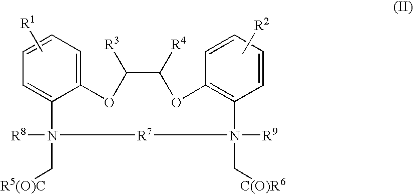
wherein:
R¹ and R² are each independently selected from one or more substituents selected from —H, -alkyl, -alkoxy, -aryl, -aryloxy, -halogen, -amino, -alkylthio, —NO₂, —COOH, —COOAlkyl, —CO-alkyl, —CN;
R¹ and R² are independently tetra-, tri- di- or mono-substitutions on each aromatic ring;
R³ and R⁴ are each independently selected from —H, -alkyl, —CH₂OH, -aryl, a sugar moiety, -polyalkyleneoxy, a water solubilising group, an antioxidant;
R⁵ and R⁶ are independently selected from —O-alkyl, —O-aryl, —S-alkyl and -amino;
R⁷ is —H (on each N atom), -alkyl, aryl, —(CH₂O)ₙ, —(CH₂CH₂O)ₙ— (n=1-5);
R⁸ and R⁹ are each independently selected from —H, -alkyl, —COOH, —COOAlkyl.

Compounds of formula (II) may also be in the form of metal salts e.g. alkali (Na⁺) and alkali earth (Ca²⁺) metal complexes.

The present inventors have found that compounds of formula (II) also have antioxidant properties. Accordingly, the present invention also provides a method for preventing or reducing the effects of oxidative stress on a substrate, the method including the step of treating the substrate with a compound of formula (II), or a pharmaceutically acceptable salt thereof.

Compounds of formula (I) or (II) are able to prevent or reduce the effects of oxidative stress on a substrate by scavenging free radicals. Accordingly, the present invention also provides a formulation for scavenging free radicals, the formulation containing the effective amount of a compound of formula (I) or (II).

The present invention also provides a pharmaceutical composition including a compound of formula (II), and a pharmaceutically acceptable excipient. The pharmaceutical composition may be used in the treatment of neurodegenerative disorders.

The present invention also provides a method for preparing a compound of formula (II) and/or a method for preparing a composition containing a compound of formula (II).

### GENERAL DESCRIPTION OF THE INVENTION

Various terms that will be used throughout the specification have meanings that will be well understood by a skilled addressee. However, for ease of reference, some of these terms will now be defined.

The term “substrate” as used throughout the specification is to be understood to mean a biological system (e.g. cell, skin), or a chemical substrate (e.g. oxygen sensitive chemicals).

The term “biological system” as used throughout the specification is to be understood to mean any cellular or multi-cellular system, and includes isolated cells to whole organisms. For example, the biological system may be a tissue in an animal or human subject suffering the effects of oxidative stress, or an entire animal or human subject suffering the effects of oxidative stress.

The term “neurodegenerative disorder” as used throughout the specification is to be understood to mean a disorder that is characterised by the premature death or loss of function of neuronal cells. Neuronal cell death or loss of function by a degenerative process is a major pathological feature of many human neurological disorders. Neuronal cell death can occur as a result of a variety of conditions including traumatic injury, ischemia, epilepsy, neurodegenerative disorders (e.g., Parkinson's disease, Huntington's disease, Alzheimer's disease, amyotrophic lateral sclerosis (ALS), multiple sclerosis, stroke, or trauma), or as a normal part of tissue development and maintenance. Several inherited disorders produce late onset neuron loss, each of which is highly specific for particular neuronal cell types.

The term “alkyl” as used throughout the specification is to be understood to mean a branched or straight chain acyclic, monovalent saturated hydrocarbon radical preferably having one to twenty carbon atoms, and more preferably having one to ten carbon atoms.

The term “alkoxy” as used throughout the specification is to be understood to mean the group “alkyl-O—”. Preferred alkoxy groups include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

The term “alkenyl” as used throughout the specification is to be understood to mean an unsaturated hydrocarbon radical which contains at least one carbon-carbon double bond and includes straight chain, branched chain and cyclic radicals.

The term “amino” as used throughout the specification is to be understood to mean a nitrogen optionally mono-, di- or tri-substituted.

The term “aryl” as used throughout the specification is to be understood to mean an aromatic monovalent carbocyclic radical having a single ring (e.g., phenyl) or two condensed rings (e.g., naphthyl), which can optionally be substituted at one or more positions on the aromatic ring.

The term “heteroaryl” as used throughout the specification is to be understood to mean an aromatic monovalent mono- or poly-cyclic radical having at least one heteroatom within the ring, e.g., nitrogen, oxygen or sulfur, wherein the aromatic ring can optionally be substituted at one or more positions on the aromatic ring.

The term “acyl” as used throughout the specification is to be understood to mean the groups alkyl-C(O)—, substituted alkyl-C(O)—, cycloalkyl-C(O)—, substituted cycloalkyl-C(O)—, aryl-C(O)—, heteroaryl-C(O)— and heterocyclic-C(O)—.

The terms “halo” or “halogen” as used throughout the specification is to be understood to mean fluoro, chloro, bromo or iodo.

The term “polyalkyleneoxy” as used throughout the specification is to be understood to mean a polyalkylether group having one or more repeating -(alkyl-O)— groups, with the alkyl preferably having 2 or 3 carbon atoms.

The term “sugar moiety” as used throughout the specification is to be understood to mean a straight chain or cyclic saccharide, especially a pentose or hexose such as glucose, fructose, mannose, and galactose or derivatives thereof.

The term “pharmaceutically acceptable salt” as used throughout the specification is to be understood to mean pharmaceutically acceptable salts of a compounds of formula (I) and (II) which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like can be used as the pharmaceutically acceptable salt.

Isolation and purification of the compounds and intermediates described herein can be effected, if desired, by any suitable separation or purification procedure such as, for example, filtration, extraction, crystallization, column chromatography, thin-layer chromatography, thick-layer (preparative) chromatography, distillation, or a combination of these procedures. Specific illustrations of suitable separation and isolation procedures can be had by reference to the examples hereinbelow. However, other equivalent separation or isolation procedures can also be used.

Derivatives of 1,2-bis(2-aminophenoxy)ethane-N,N,N′,N′-tetraacetic acid (BAPTA) according to formula (I) or (II) were synthesised and found to have free radical scavenging properties. These derivatives may act as prodrugs of the physiologically active acids or salts of BAPTA. Without being bound by a specific theory on the mode of action of compounds of formula (I) or (II), the present inventors postulate that these compounds act as cell membrane permaeable covalent conjugates of BAPTA. On exposure to intracellular enzyme activity, one or more covalent bonds are broken in the conjugates to release the active species, which is believed to be BAPTA or a salt thereof.

To the best of the inventor's knowledge, the free radical scavenging properties of compounds of formula (I) or (II) have not previously been investigated or identified. Accordingly, these compounds represent a new class of free radical scavenging or antioxidant compounds that can be utilised in the treatment of neurological and other disorders associated with oxidative stress.

Evidence for an association of oxidative stress has been made in a large number of medical conditions. Oxidative stress is likely to play an especially significant role in chronic, degenerative disorders or conditions that accompany the ageing process. These include conditions such as neurodegenerative disorders (eg. Alzheimer's, Parkinson's, Huntington's etc), neoplastic diseases, central nervous system disorders, vascular disorders, diabetic complications, ageing and ischemic tissue injury.

Genetic or post-translational alterations of the free radical scavenging enzyme Cu/Zn superoxide dismutase (SOD) within motor neurons interfere with its function and render motor neurons vulnerable to free radical attack. Free radical scavengers such as vitamin E have previously been shown to reduce neurodegeneration and prolong the life of transgenic mice that develop motor neuron disease. Hence, the free radical scavenging compounds of formula (I) or (II) can be used to treat neurological disorders and traumatic injuries of the nervous system.

As previously mentioned the present invention provides a method for preventing or reducing the effects of oxidative stress on a substrate, the method including the step of treating the substrate with a compound of formula (I), or a pharmaceutically acceptable salt thereof: 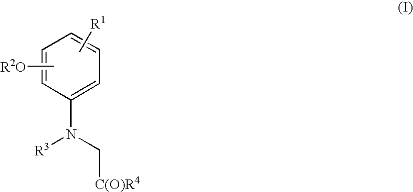
wherein:
R¹ is one or more substituents selected from —H, -alkyl, -alkoxy, -aryl, -aryloxy, -halogen, -amino (mono-, di- and tri-substituted), -alkylthio, —NO₂, —COOH, —COOAlkyl, —CO-alkyl, —CN;
R² is one or more substituents selected from —H, -alkyl, —(CH₂CH₂O)ₙ—R⁵, a sugar moiety;
R³ is —H, -alkyl, -aryl, -alkylOR⁶, -alkylC(O)R⁶;
R⁵ is selected from —H, -alkyl, -aryl; and
R⁴ and R⁶ are independently selected from —OH, —O-alkyl, —O-polyalkyleneoxy, —O-aryl, —OC(O)O-alkyl, —S-alkyl and -amino.

The present invention also provides a method for preventing or reducing the effects of oxidative stress on a biological system, the method including the step of treating the biological system with a compound of formula (I), or a pharmaceutically acceptable salt thereof: 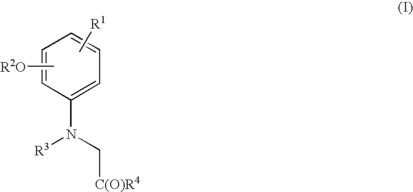
wherein:
R¹ is one or more substituents selected from —H, -alkyl, -alkoxy, -aryl, -aryloxy, -halogen, -amino (mono-, di- and tri-substituted), -alkylthio, —NO₂, —COOH, —COOAlkyl, —CO-alkyl, —CN;
R² is one or more substituents selected from —H, -alky, —(CH₂CH₂O)ₙ—R⁵, a sugar moiety;
R³ is —H, -alkyl, -aryl, -alkylOR⁶, -alkylC(O)R⁶;
R⁵ is selected from —H, -alkyl, -aryl; and
R⁴ and R⁶ are independently selected from —OH, —O-alkyl, —O-polyalkyleneoxy, —O-aryl, —OC(O)O-alkyl, —S-alkyl and -amino.

For both methods, in the compound of formula (I) R² is preferably —CH₂CH₂O—R⁵, R³ is preferably —CH₂C(O)R⁶, R⁴ and R⁶ are preferably one or more of —O-methyl, —O-ethyl, —OCH₂CH₂OCH₂CH₂OCH₂CH₂OCH₃ or —OC(O)CH₃.

Alternatively or in addition, for both methods R⁵ preferably has the following formula: 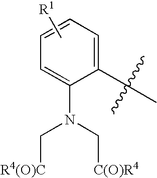
wherein:
R¹ is one or more substituents selected from —H, -alkyl, -alkoxy, -aryl, -aryloxy, -halogen, -amino, -alkylthio, —NO₂, —COOH, —COOAlkyl, —CO-alkyl, —CN; and
R⁴ is selected from —OH, —O-alkyl, —O-polyalkyleneoxy, —O-aryl, —OC(O)O-alkyl, —S-alkyl and -amino.

Compounds of formula (I) and (II) according to Table 1 have been synthesised and have all shown to have anti-oxidant properties as assessed using the methods described herein.

TABLE 1

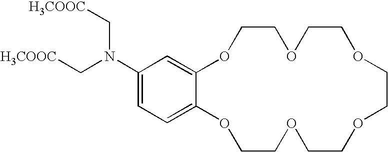

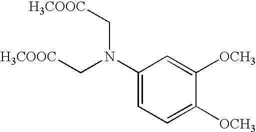

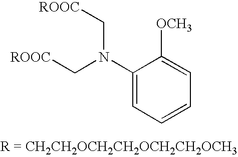

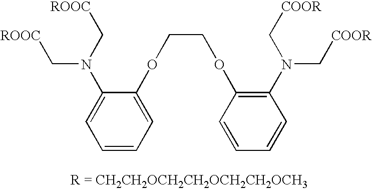

In another specific embodiment of the invention the compound of formula (I) has the following formula: 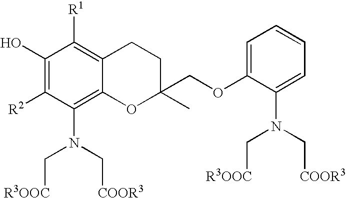
wherein
R¹ and R² are independently selected from —H, -alkyl, -alkoxy, -aryl, -aryloxy, -halogen, -amino, -alkylthio, —NO₂, —COOH, —COOAlkyl, —COAlkyl, —CN; and
R³ is selected from —OH, —Oalkyl, —OAryl, —Salkyl, amino, a sugar moiety, -polyalkyleneoxy, and a water solubilising group.

In addition, compounds of formula (II), or pharmaceutically acceptable salts thereof may be used in the methods of the invention. 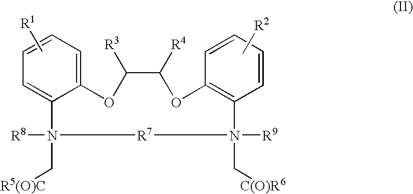
wherein:
R¹ and R² are each independently selected from one or more substituents selected from —H, -alkyl, -alkoxy, -aryl, -aryloxy, -halogen, -amino, -alkylthio, —NO₂, —COOH, —COOAlkyl, —CO-alkyl, —CN;
R¹ and R² are independently tetra-, tri- di- or mono-substitutions on each aromatic ring;
R³ and R⁴ are each independently selected from —H, -alkyl, —CH₂OH, -aryl, a sugar moiety, -polyalkyleneoxy, a water solubilising group, an antioxidant;
R⁵ and R⁶ are independently selected from —O-alkyl, —O-aryl, —S-alkyl and -amino;
R⁷ is —H (on each N atom), -alkyl, aryl, —(CH₂O)ₙ—, —(CH₂CH₂O)ₙ— (n=1-5);
R⁸ and R⁹ are each independently selected from —H, -alkyl, —COOH, —COOAlkyl.

In some cases it may be preferable to increase the aqueous solubility of the compounds of the invention. For aqueous applications, it is preferred that at least one of the R groups (for example R⁴ in compounds of formula (I) or R³/R⁴ in compounds of formula (II)) includes a water-solubilising group, such as sulfonate, sulfate, carboxylate, hydroxyl, amino, ammonium, sugar, straight chain or cyclic saccharides, ascorbate groups, alkyl chains substituted with —OH at any position, glycols, including polyethylene glycols, polyether, boronate and the like, to enhance the solubility or transport of the control agent.

The capacity of a particular compound to scavenge free radicals can be determined using a method in which the reduction of 3-(4,5-dimethylthiazole-2-yl)-2,5-diphenyltetrazoliumbromide (MTT) by the test compound is measured.

Alternatively, or in addition, the capacity of a particular compound to scavenge free radicals can be determined by measuring the efficacy of a test compound in the oxidation of linoleic acid by hydrogen peroxide in the presence of the test compound. The results indicate that compounds of formula (I) and (II) act as potent antioxidants with the same efficacy as that displayed by vitamin E (a known antioxidant). Similar results were obtained upon the addition of varying concentrations of calcium ions within the assay.

In addition to the free radical scavenging activity of the BAPTA analogues described, the compounds also act as calcium buffers. Accordingly, these analogues have a binary action in that they scavenge free radicals as well as buffering calcium.

Besides oxidative stress based pathways, there is considerable evidence that excitotoxic pathways can also lead to neurodegeneration. The functioning of neurons and synaptic activity are heavily dependent on calcium ions. Excitotoxicity can be triggered by excessive levels of glutamate that over-stimulates ionotropic receptors leading to excessive influx of Ca²⁺. This calcium overload activates proteases and nucleases resulting in neuronal death. The inability of endogenous Ca²⁺ buffering proteins to deal with pathologic intracellular Ca²⁺ loads renders neurons particularly vulnerable to calcium toxicity. A variety of disorders, such as those associated with ischaemia, injury and neurodegenerative disorders are initiated via dysregulation of Ca²⁺ homeostasis.

Moreover, evidence from rodent species suggests that changes in the neuronal calcium homeostasis coincide with aging of the brain in general, and may be correlated with age-related decline in cognitive functions. The experimental evidence has led to the suggestion that changed calcium homeostasis in aged neurons may be a contributing factor to some memory deficits caused by aging. According to the present invention, a therapeutically effective amount of a free radical scavenger and a calcium buffer can be administered to an animal or human to treat a disease state that is associated with calcium toxicity and oxidative stress. For these purposes, compounds of formula (I) or (II) may be administered using any suitable administration protocol.

Compounds of formula (I) or (II) (or pharmaceutically acceptable salts thereof) may be prepared as pharmaceutical compositions with pharmaceutically acceptable excipients, carriers, diluents, permeation enhancers, solubilizers and adjuvants. Suitable pharmaceutically acceptable excipients include vehicles and carriers capable of being coadministered with compounds of formula (I) or (II) to facilitate the performance of their intended function. The use of such media for pharmaceutically active substances is known in the art.

Some examples of suitable excipients include lactose, dextrose, sucrose, sorbitol, mannitol, starches, gum acacia, calcium phosphate, alginates, tragacanth, gelatin, calcium silicate, microcrystalline cellulose, PEG, polyvinylpyrrolidone, cellulose, water, sterile saline, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavouring agents. The compositions can be formulated so as to provide quick, sustained or delayed release of the active ingredient after administration to the patient.

One or more compounds of formula (I) or (II) may be administered alone or in combination with other therapeutic agents (e.g. other agents for treatment of neurodegenerative disorders), carriers, adjuvants, permeation enhancers, and the like. The compositions may be formulated using conventional techniques such as those described in ‘Remington's Pharmaceutical Sciences’, Mace Publishing Co., Philadelphia, Pa. 17th Ed. (1985) and ‘Modern Pharmaceutics’, Marcel Dekker, Inc. 3rd Ed. (G. S. Banker & C. T. Rhodes, Eds.). Pharmaceutically acceptable salts of compounds of formula (I) may be prepared using standard procedures known to those skilled in the art of formulation chemistry.

The compounds of formula (I) or (II) may be administered by any of the accepted modes of administration of therapeutic agents, for example, by orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The most suitable route will depend on the nature and severity of the condition being treated. The term parenteral as used herein includes subcutaneous, intravenous, intramuscular, intraperitoneal, intrathecal, intraventricular, intrasternal, and intracranial injection or infusion techniques. For parenteral administration, the compositions can be in the form of sterile injectable solutions and sterile packaged powders.

When administered orally, the composition will usually be formulated into unit dosage forms such as tablets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

The compositions are preferably formulated in a unit dosage form in physically discrete units suitable as unitary dosages for human subjects and other mammals, each unit containing a predetermined quantity of active material calculated to produce the desired therapeutic effect, in association with a suitable pharmaceutical excipient.

Compounds of formula (I) or (II), or their pharmaceutically acceptable salts, are administered in a therapeutically effective amount. The amount of the compound actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

Compounds of formula (I) or (II) may be administered at a dosage of between 1 and 50 mg/kg. Doses of 1, 10, 100 and 1000 mg/kg may be administered three times per week intraperitoneally. It is also envisaged that formulations containing the compounds of formula (I) or (II) formulations could be administered orally.

Antioxidant compounds of formula (I) or (II) may be used in as anti-aging agents and therefore compositions containing one or more of these compounds may be used cosmetically and applied topically. For topical use, the compositions can be in the form of emulsions, creams, jelly, solutions, ointments containing, for example, up to 5% by weight of the active compound.

Besides treatment with compounds of formula (I) or (II), the method of the present invention may involve administration of a first agent that is free radical scavenger and a second agent that is a calcium buffer. Suitable free radical scavengers include carotenoids, limonoids, phytosterols, flavonoids, anthocyanidins, catechins, isoflavones, oligomeric proanthocyanidins, isothiocyanates, dithiolthiones, sulforaphane, isoprenoids, tocotrienols, tocopherols (e.g. vitamin E), lipoic acid, ubiquinone, ascorbates (e.g. vitamin C), 2,3-dihydro-1-benzofuran-5-ols, chromanones, C60 and trolox. Suitable calcium buffers include derivatives of 15-crown-5, 18-crown-6, EDTA and BAPTA.

### BRIEF DESCRIPTION OF THE FIGURES

FIG. 1 is a time vs absorbance plot at 234 nm for the oxidation of linoleic acid in the presence of an antioxidant of the present invention. “MJM carboxylic” as used in the plot refers to compound (4) whilst “MJM ester” refers to compound (3).

FIG. 2 is a plot of the oxidant stress-induced cell death of neonatal mouse fibroblasts exposed to paraquat. Cells (5×10³ per well) were treated overnight with freshly prepared paraquat or peroxynitrite. Culture medium from wells was assayed for lactate dehydrogenase (LDH) release as a marker of cytotoxicity. High doses of paraquat but not peroxynitrite induced significant cell death. Values represent means±SEMs of triplicate wells, *P<0.05 difference from untreated cells. The x-axis represents concentration of Paraquat and Peroxynitrite in μM.

FIG. 3 is a plot showing the effect of (S)-5-fluorowillardiine (“(S)-5-FW”) on survival (MTT reduction) of NSC-34_{D} motor neuron cell line. These cells were cultured at 1×10⁴ cells/well for 48 hours in DMEM/F-12 with 1% (v/v) FCS and then exposed to various concentrations of (S)-5-FW for 72 hours. MTT solution (0.5 mg/ml) was added to cultures for 2 hours, cells were solubilised and MTT reduction quantified as a percentage of treated cells. Statistically significant differences from control (zero (S)-5-FW) were defined using one-way ANOVA (*P<0.05, ***P<0.001). It can be seen that (S)-5-FW elicited significant cell death in concentration dependent manner with the maximal effect at a concentration of 1000 μM [(S)-5-FW].

FIG. 4 is a plot showing the body weights of SODI^{693A} transgenic mice co-treated with compound (3). A statistically significant reduction (asterisk) in the weight associated with muscle atrophy was observed at postnatal day 116 in vehicle (A) group. This was delayed to postnatal day 130 in the remaining groups i.e. LIF+(3) (B), PNAG3+(3) (C) and LIF+PNAG3+(3) (D). There were 10 mice in each group (5 male, 5 female), mean+SEM *P<0.05.

FIG. 5 is a plot of the locomotor performance and survival in the SODI^{693A} transgenic mice in Vehicle (VEH), LIF+ compound (3), PNAG3+ compound (3) and LIF+PNAG3+ compound (3) groups (n=10 per group). Histogram 'A shows a significant difference in LIF+ compound (3), PNAG3+ compound (3) and LIF+PNAG3+ compound (3) groups compared to Vehicle group. There was no significant difference between these three groups. A similar significant improvement in the three treated groups was also observed in the bar grab test (B). Kaplan-Meier survival curve of treated mice also shows a significant increase in lifespan in the LIF+ compound (3), PNAG3+ compound (3) and LIF+PNAG3+ compound (3) groups compared to the vehicle group (C).

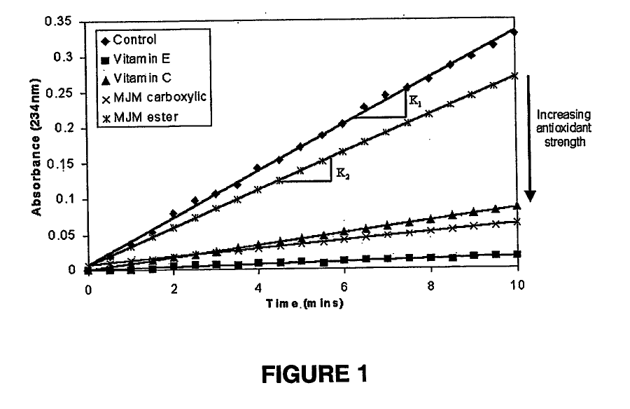

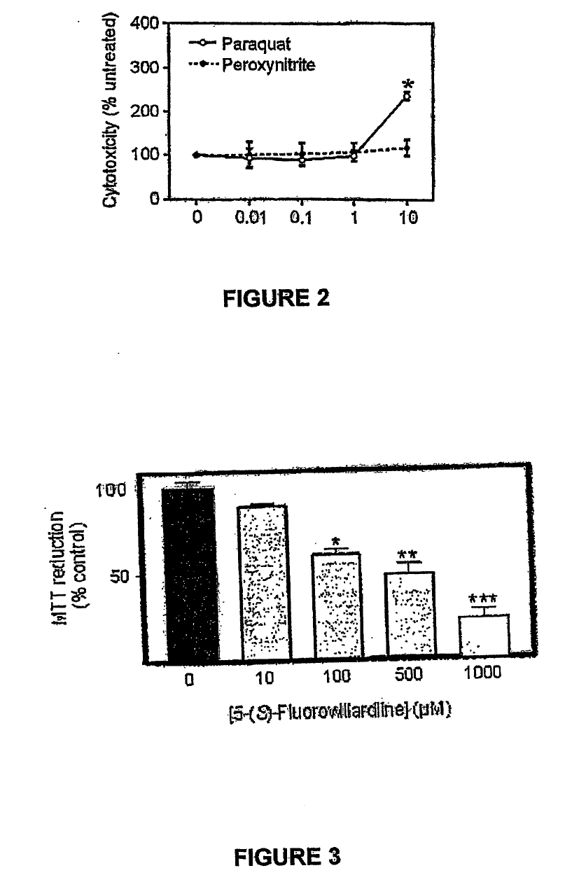

### DESCRIPTION OF PREFERRED EMBODIMENTS OF THE INVENTION

Reference will now be made to examples that embody the above general principles of the present invention. However, it is to be understood that the following description is not to limit the generality of the above description.

Synthesis of Compounds of Formula (I) or (II)

### EXAMPLE 1

### Synthesis of bis[2-(bis-ethoxycarbonylmethyl)aminophenoxy]ethane (“compound (3)”) and bis(o-aminophenoxy)ethane-N,N,N′,N′-tetraacetic acid (BAPTA) (“compound (4)”)

Bis[2-(bis-ethoxycarbonylmethyl)aminophenoxy]ethane (3) and bis(o-aminophenoxy)ethane-N,N,N′,N′-tetraacetic acid (4) were prepared using literature procedures (see Tsien, R. Y., J. Am. Chem. Soc., 1980, 19, 2396-2404 and Grynkiewicz, G.; Poenie, M. and Tsien, R. Y., J. Biol. Chem., 1985, 260(6), 3440-3460).

### EXAMPLE 2

### Synthesis of N,N-bis(acetic acid)o-anisidine (5)

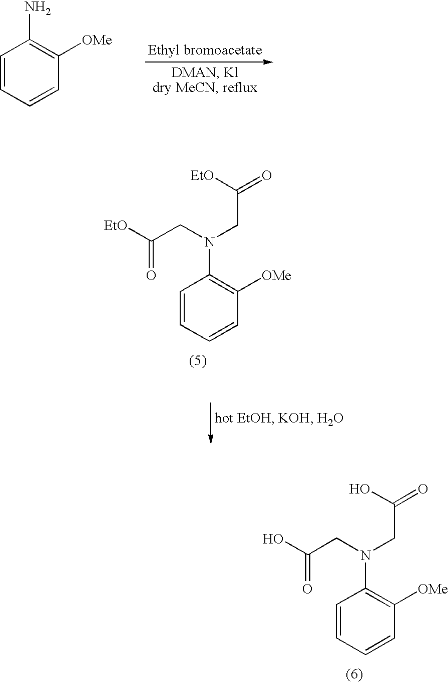

Synthesis of N,N-bis(ethoxycarbonylmethyl)o-anisidine (5): 0.92 mL (8.12 mmol) of o-Anisidine was mixed with 1.2 equivalents of Proton Sponge (2.09 g), 0.14 equivalents of potassium iodide (189 mg) and 2.4 equivalents of ethylbromoacetate (2.16 mL) in dry acetonitrile and refluxed overnight. The reaction mixture was diluted with toluene and filtered, the filtrate washed with 2M HCl (×2) and H₂O, dried over Na₂SO₄, filtered and the solvent removed under reduced pressure. The crude product was purified by column chromatography (30% EtOAc/hexane) to give 1.10 g (3.69 mmol) of (5) as a yellow oil (45% yield). ¹H NMR δ(300 MHz, CDCl₃): 6.88, m, 4H, Ar—H; 4.18, q, 4H, J 7.17 Hz, —O—CH₂—CH₃; 4.13, s, 4H, N—CH₂—; 3.80, s, 3H, —O—CH₃; 1.25, t, 6H, J 7.17 Hz, —O—CH₂—CH₃. ¹³C NMR δ(75 MHz, CDCl₃): 171.2, C═O; 151.2, C1; 138.6, C2; 122.2/120.8/119.0/111.9, C3/C4/C5/C6; 60.6, —O—CH₂—CH₃; 55.5, —O—CH₃; 54.0, N—CH₂—; 14.3, —O—CH₂—CH₃. MS (ESI, +ve): m/z 296.1 [M plus H]⁺, 318.1 [M plus Na]⁺, 613.3 [2M plus Na]⁺.

N,N-bis(acetic acid)o-anisidine (6): In air, 200 mg (0.677 mmol) of (5) was dissolved in the minimum amount of warm ethanol (7 mL) and treated with 2.1 equivalents of potassium hydroxide (80 mg) as a concentrated aqueous solution. After gentle warming for 15 min, ethanol was removed under reduced pressure and the residue redissolved in H₂O (20 mL) and cooled in an ice bath. Concentrated HCl was slowly added to pH 2. No precipitate formed on addition of HCl, so the product was extracted into EtOAc and the solvent removed under reduced pressure. Crude product was recrystallized from EtOAc/hexane (×2) to give 40 mg (0.207 mmol) of (6) as a pale tan solid (31% yield). ¹H NMR δ(300 MHz, CDCl₃/DMSO): 6.77, m, 4H, Ar—H, 3.90, s, 4H, N—CH₂—; 3.68, s, 3H, —O—CH₃. ¹³C NMR δ(75 MHz, CDCl₃/DMSO): 173.5, C—O; 151.5, C1; 138.0, C2; 122.9/120.7/119.7/111.7, C3/C4/C5/C6; 55.5, N—CH₂—; 55.3, —O—CH₃. MS (ESI, +ve): m/z 240.1 [M plus H]⁺, 262.1 [M plus Na]⁺.

### EXAMPLE 3

### Synthesis of 4-amino-1,2-dimethoxybenzene-N,N-diacetyl ethyl ester (10) and 4-amino-1,2-dimethoxybenzene-N,N-diacetic acid (11)

### Synthesis of 4-nitro-1,2-dimethoxybenzene (8)

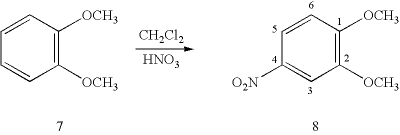

Concentrated nitric acid (2 ml) was added to a solution of veratrole (7) (2.5 ml, 19.6 mmol) and dichloromethane (50 ml) and allowed to stir at room temperature for 24 hours. The organic layer was then washed with water (3×50 ml), dried over sodium sulfate, filtered and the solvent removed under reduced pressure. The isolated product solidified upon standing to give 3.5 g of (8) as a yellow solid (0.02 mol, 98% yield).

Melting point: 90-94° C. (Literature mp: 97-98° C.).²⁹

¹H n.m.r. (300 MHz, CDCl₃): δ 7.92 (dd, 1H, J 8.9 Hz, J 2.6 Hz, Ar—H); 7.75 (d, 1H, J 2.6 Hz, Ar—H); 6.91 (d, 1H, J 8.9 Hz, Ar—H, 3.98 (s, 3H, [C²]—OCH₃); 3.97 (s, 3H, [C¹]—OCH₃). ¹³C n.m.r. (75 MHz, CDCl₃): δ 153.6, (C⁴); 147.9, (C²); 140.6, (C¹); 116.8, (C⁵); 108.9, (C⁶); 105.5, (C³); 55.5, ([C²]—OCH₃); 55.3, ([C¹]—OCH₃). IR (Nujol): 1586, m; 1500, s; 1345, s; 1280, s; 804, m. Mass spectrum (ESI, +ve): m/z 183.8 [M]⁺.

### Synthesis of 4-amino-1,2-dimethoxybenzene (9)

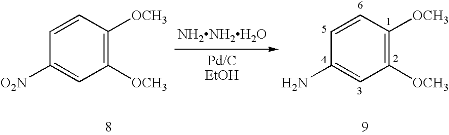

4-Nitro-1,2-dimethoxybenzene (8)] (1.03 g, 5.62 mmol) was dissolved in 95% (v/v) ethanol (60 ml) and hydrogenated with hydrazine hydrate (30 ml) over palladium on charcoal (0.11 g). The reaction was refluxed for 24 hours. The hot solution was filtered through celite and solvent removed under reduced pressure. Water was added and the product was extracted into dichloromethane, dried over sodium sulfate, filtered and solvent removed under reduced pressure to give a viscous liquid, which was then placed on ice to give 0.80 g of (9) as a white powder (5.22 mmol, 93% yield).

Melting point: 82-85° C. (Literature mp: 83-87° C.).³⁰ Decomposition at: 205° C.

¹H n.m.r. (300 MHz, CDCl₃): δ 6.66 (d, 1H, J 8.4 Hz, Ar—H); 6.26 (d, 1H, J 2.6 Hz, Ar—H), 6.18 (dd, 1H, J 8.4 Hz, 2.6 Hz, Ar—H); 3.77 (s, 3H, [C¹]—OCH₃), 3.76 (s, 3H, [C²]—OCH₃), 3.38 (br s, 2H, NH₂). ¹³C n.m.r. (75 MHz, CDCl₃): δ 148.9, (C¹); 141.1, (C²); 139.8, (C⁴); 112.3, (C⁶); 105.5, (C⁵); 99.8, (O); 55.7, ([C¹]—OCH₃); 54.7, ([C²]—OCH₃). IR (Nujol): 3380, br s; 1596, m; 1514, s; 11463, s; 1236, s; 849, m. Mass spectrum (ESI, +ve): m/z 153.8 [M]⁺.

### Synthesis of 4-amino-1,2-dimethoxybenzene-N,N-diacetyl ethyl ester (10)

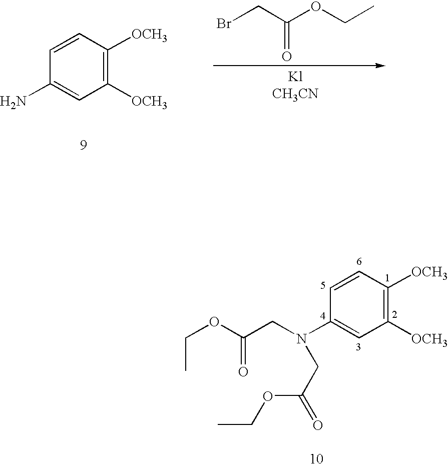

A mixture of 4-amino-1,2-dimethoxybenzene (9) (0.15 g, 0.95 mmol), potassium iodide (0.32 g) and ethyl bromoacetate (0.5 ml) was refluxed for 48 hours in dry acetonitrile (5 ml) under a nitrogen atmosphere. The solution was then cooled, diluted with toluene (5 ml) and filtered. The collected filtrate was washed with 2M hydrochloric acid (2×20 ml) and water (2×20 ml), dried over sodium sulfate, filtered and the solvent removed under reduced pressure to give 0.14 g of (10) as a brown oil (0.43 mmol, 45% yield).

¹H n.m.r. (300 MHz, CDCl₃): δ 6.74 (d, 1H, J 8.7 Hz, Ar—H); 6.34 (d, 1H, J 2.8 Hz, Ar—H), 6.20 (dd, 1H, J 8.7 Hz, 2.8 Hz, Ar—H); 4.21 (q, 4H, J 7.1 Hz, —O—CH₂—CH₃); 4.12 (s, 4H, —N—CH₂—); 3.81 (s, 3H, [C¹]—O—CH₃); 3.77 (s, 3H, [C²]—O—CH₃); 1.25 (t, 6H, J 7.1 Hz, —O—CH₂—CH₃). ¹³C n.m.r. (75 MHz, CDCl₃): δ 169.9, (C═O); 148.8, (C¹); 141.7, (C²); 141.6, (C⁴); 112.0, (C⁶); 104.4, (C⁵); 98.7, (C³); 60.2, (—O—CH₂—CH₃); 55.6, ([C¹]—OCH₃); 54.9, ([C²]—OCH₃); 53.5, (—N—CH₂—); 13.3, (—O—CH₂—CH₃). IR (Nujol): 2978, m; 2853, w; 1744, s; 1520, s; 1189, s. Mass spectrum (ESI, +ve): m/z 326.3 [M]⁺; 348.3 [M+Na]⁺.

### Synthesis of 4-amino-1,2-dimethoxybenzene-N,N-diacetic acid (11)

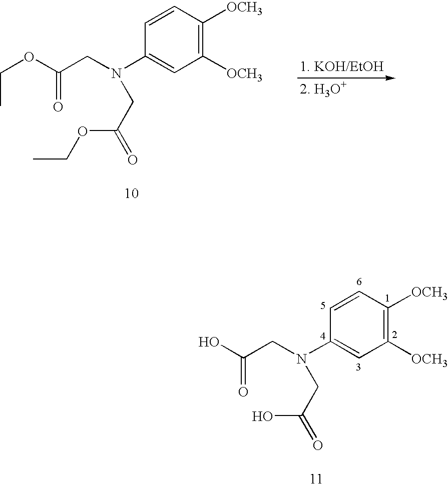

The ester compound (10) was saponified to its respective potassium salt by dissolution in warm ethanol (10 ml) and the addition of a dilute aqueous solution (0.2 g in 20 ml) of potassium hydroxide (5 ml). The solution was warmed for 5 minutes and the ethanol removed under reduced pressure. The carboxylic acid was obtained by dropwise addition of 6M hydrochloric acid whilst stirring. The product was extracted into ethyl acetate (20 ml), and the solvent removed under reduced pressure to give 30.2 mg of (11) as a dark red/brown solid (0.11 mmol, 24%).

¹H n.m.r. (300 MHz, d₆-DMSO): δ 6.79 (d, 1H, J 8.8 Hz, Ar—H); 6.21 (d, 1H, J 2.9 Hz, Ar—H); 6.01 (dd, 1H, J 8.8 Hz, 3.0 Hz, Ar—H); 4.07 (s, 4H, —N—CH₂—); 3.70 (s, 3H, [C¹]—OCH₃); 3.62 (s, 3H, [C²]—OCH₃). ¹³C n.m.r. (75 MHz, d₆-DMSO): δ 172.1, (C═O); 149.1, (C²); 142.5, (C¹); 140.5, (C⁴); 113.8, (C⁶); 102.8, (C⁵); 97.9, (C³); 56.0, ([C¹]—OCH₃); 54.9, ([C²]—OCH₃); 52.9, (—N—CH₂—).

### EXAMPLE 4

### Synthesis of 4-amino[N,N-diacetyl ethyl ester]benzo-18-crown-6 (15) and 4-amino[N,N-diacetic acid]benzo-18-crown-6 (16)

### Synthesis of 4-nitrobenzo-18-crown-6 (13)

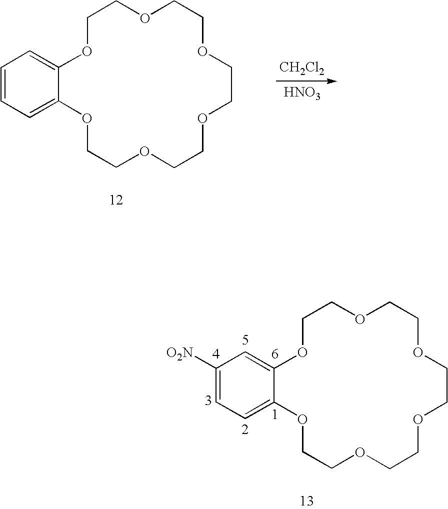

Concentrated nitric acid (0.25 ml) was added to a stirring solution of benzo-18-crown-6 (12) (0.25 g, 0.81 mmol) in dichloromethane (15 ml). The solution was allowed to stir at room temperature for 24 hours. The organic layer was washed with water (3×30 ml), dried over sodium sulfate, filtered and the solvent removed under reduced pressure. The isolated oily product solidified upon standing to give 0.28 g of (13) as a yellow crystalline solid (0.78 mmol, 96% yield). ¹H n.m.r. of the crude indicated the presence of product and also dichloromethane. The product was allowed to dry for another 24 hours before its employment in subsequent reactions.

Melting point: 82-83° C. (Literature mp: 80-81° C.).³¹ Decompostion at: 312° C.

¹H n.m.r. (300 MHz, CDCl₃): 7.88 (dd, 1H, J8.9 Hz, 2.6 Hz, Ar—H; 7.74 (d, 1H, J 2.6 Hz, Ar—H); 6.89 (d, 1H, J 8.9 Hz, Ar—H; 4.24 (m, 4H, OCH₂); 3.95 (m, 4H, OCH₂); 3.77 (m, 4H, OCH₂); 3.72 (m, 4H, OCH₂); 3.68 (s, 4H, OCH₂). ¹³C n.m.r. (75 MHz, CDCl₃): δ 154.3, (C⁴); 148.4, (C²); 141.3, (C¹); 117.9, (C⁵); 111.2, (C⁶); 108.1, (C³); 70.99, (OCH₂); 70.98, (OCH₂); 70.8, (OCH₂); 70.7, (OCH₂); 70.60, (OCH₂); 70.57, (OCH₂); 69.23, (OCH₂); 69.17, (OCH₂); 69.1, (OCH₂). IR (Nujol): 1587, m; 1520, s; 1464, s; 1338, s, 1276, s; 1128, s; 864, w. Mass spectrum (ESI, +ve): m/z 358.1 [M]⁺; 381.1 [M+Na]⁺.

### Synthesis of 4-aminobenzo-18-crown-6 (14)

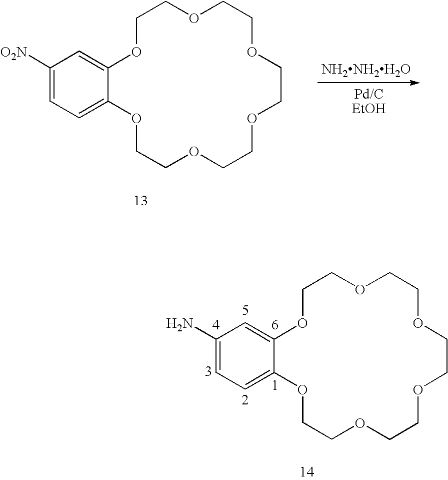

4-Nitrobenzo-18-crown-6 (13) (0.40 g, 1.11 mmol) was dissolved in 95% (v/v) ethanol (25 ml) and hydrogenated with hydrazine hydrate (12 ml) over palladium on charcoal (40 mg). The reaction was refluxed for 24 hours. The hot solution was filtered through celite and the solvent removed under reduced pressure. Water was added and the product extracted into dichloromethane, dried over sodium sulfate, filtered and solvent removed under reduced pressure to give a viscous liquid, which was then placed on ice to give 0.28 g of (14) as a white powder (0.86 mmol, 84% yield).

¹H n.m.r. (300 MHz, CDCl₃): δ 6.74 (d, 1H, J 8.4 Hz, Ar—H); 6.29 (d, 1H, J 2.6 Hz, Ar—H); 6.21 (dd, 1H, J 8.4 Hz, 2.6 Hz, Ar—H); 4.09 (m, 4H, OCH₂); 3.89 (m, 4H, OCH₂), 3.73 (m, 8H, OCH₂); 3.68 (s, 4H, OCH₂), 3.46 (br s, 2H, NH₂). ¹³C n.m.r. (75 MHz, CDCl₃): δ 149.0, (C¹); 140.7, (C²); 140.4, (C); 115.5, (C⁶); 106.1, (C⁵); 101.5, (C³); 69.7, (OCH₂); 69.6, (OCH₂); 69.6, (OCH₂); 69.5, (OCH₂); 69.0, (OCH₂); 68.8, (OCH₂); 68.6, (OCH₂); 67.5, (OCH₂). IR (Nujol): 3585, m; 3362, m; 1622, m; 1594, m; 1100, s. Mass spectrum (ESI, +ve): m/z 328.1 [M]⁺; 351.1 [M+Na]⁺.

### Synthesis of 4-amino[N,N-diacetyl ethyl ester]benzo-18-crown-6 (15)

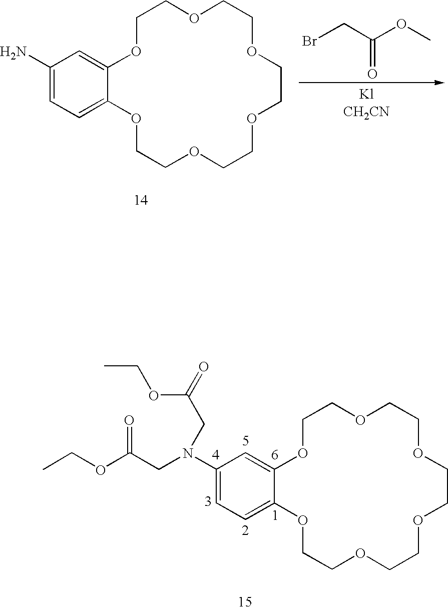

A mixture of 4-aminobenzo-18-crown-6 (14) (0.28 g, 0.84 mmol), potassium iodide (0.28 g) and ethyl bromoacetate (0.2 ml) was refluxed for 48 hours in dry acetonitrile (6 ml) under a nitrogen atmosphere. The solution was cooled, diluted with toluene (10 ml) and filtered. The collected filtrate was washed with 2M hydrochloric acid (3×20 ml) and water (2×20 ml), dried over sodium sulfate, filtered and solvent removed under reduced pressure to give 84 mg of (15) as a brown oil (0.17 mmol, 20% yield).

¹H n.m.r. (300 MHz, CDCl₃): δ 6.79 (d, 1H, J 8.7 Hz, Ar—H, 6.29 (d, 1H, J 2.9 Hz, Ar—H); 6.16 (dd, 1H, J 8.7, 2.7 Hz, Ar—); 4.20 (q, 4H, J 7.1 Hz, —OCH₂—CH₃); 4.10 (m, 4H, OCH₂); 4.08 (s, 4H, N—CH₂—); 3.89 (m, 4H, OCH₂); 3.72 (m, 8H, OCH₂); 3.67 (s, 4H, OCH₂); 1.26 (t, 6H, J 7.1 Hz, —O—CH₂—CH₃). ¹³C n.m.r. (75 MHz, CDCl₃): δ 170.0, (C═O); 148.7, (C¹); 142.7, (C²); 140.7, (C⁴); 115.3, (C⁶); 104.7, (C⁵); 100.5, (C³); 69.7, (OCH₂); 69.6, (OCH₂); 69.6, (OCH₂); 69.5, (OCH₂); 69.5, (OCH₂); 68.7, (OCH₂); 68.6, (OCH₂); 67.8, (OCH₂); 60.1, (—O—CH₂—CH₃); 53.1, (N—CH₂—); 13.3, (—O—CH₂—CH₃). IR (Nujol): 2876, m; 2362, w; 1744, s; 1615, m; 1520, s; 1452, m; 1183, m. Mass spectrum (ESI, +ve): m/z 500.4 [M]⁺; 522.5 [M+Na]⁺.

### Synthesis of 4-amino[N,N-diacetic acid]benzo-18-crown-6 (16)

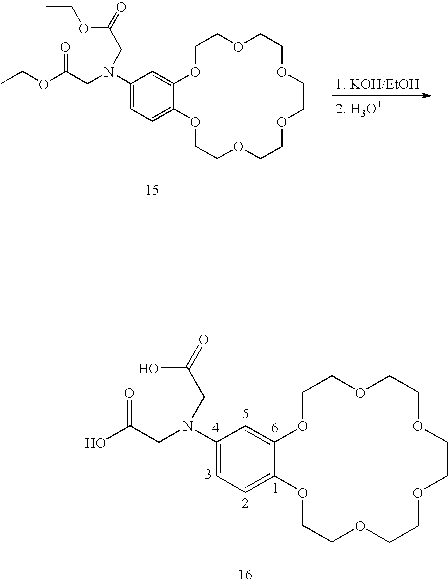

The ester compound (15) was saponified to its respective potassium salt by dissolution in warm ethanol (10 ml) and the addition of a dilute aqueous solution (0.2 g in 20 ml) of potassium hydroxide (5 ml). The solution was warmed for 5 minutes and ethanol removed under reduced pressure. The carboxylic acid was obtained by dropwise addition of 6M hydrochloric acid whilst stirring. The product was then extracted into ethyl acetate (20 ml), dried over sodium sulfate, filtered, and solvent removed under reduced pressure to give 12.5 mg of (16) as a yellow solid (0.03 mmol, 21% yield).

¹H n.m.r. (300 MHz, d₆-DMSO): δ 6.83 (d, 1H, J 8.7 Hz, Ar—H, 6.25 (d, 1H, J 2.4, Ar—H, 6.03 (dd, 1H, J 8.7 Hz, 2.8 Hz, Ar—); 4.07 (s, 4H, N—CH₂—); 4.01 (m, 4H, OCH₂); 3.77 (m, 4H, OCH₂); 3.58 (m, 8H, OCH₂); 3.55 (s, 4H, OCH₂).

Uses of Compounds of Formula (I) or (II)

### EXAMPLE 5

In Vitro Antioxidant Assay for Compounds of Formula (I) or (II)

Lipid oxidation can usually be prevented by the addition free radical scavengers (FRS), more commonly known as antioxidants. Their role is to turn reactive and harmful molecules such as hydroxy, nitroxy and superoxide radicals into innocuous molecules that can be passed or converted by biological processes. One of the main pathways that lead to apoptosis in cells is oxidative stress. This event is a product of the action of these harmful radicals on the cell walls and membranes, leading to their breakdown. In terms of the antioxidant evaluation (Scheme 1), MPH was utilised as a free radical initiator generating superoxide, and the efficiency was calculated based on the formation of a conjugated diene hydroperoxide generated through the oxidation of linoleic acid in the presence and absence of a free radical scavenger. The conjugated diene hydroperoxide mimics the products of cellular oxidative stress imposed on the cell wall. The antioxidant can act at any stage of the reaction to stop or slow down the formation of the conjugated diene by reacting with the appropriate radicals (Scheme 1).

A convenient test to determine the efficiency of antioxidants in aqueous systems was setup using 2,2′-azobis(2-amidinopropane).2HCl (MPH) as a free radical initiator (Scheme 1). The production of conjugated diene hydroperoxide (LOOH) generated through the oxidation of linoleic acid in an aqueous system at 37° C. is monitored at 234 nm for 15 minutes using a Cary 100 UV-Vis spectrophotometer. The efficiency of the antioxidant is measured by its ability to quench free radicals and hence slow or stop oxidation of linoleic acid.

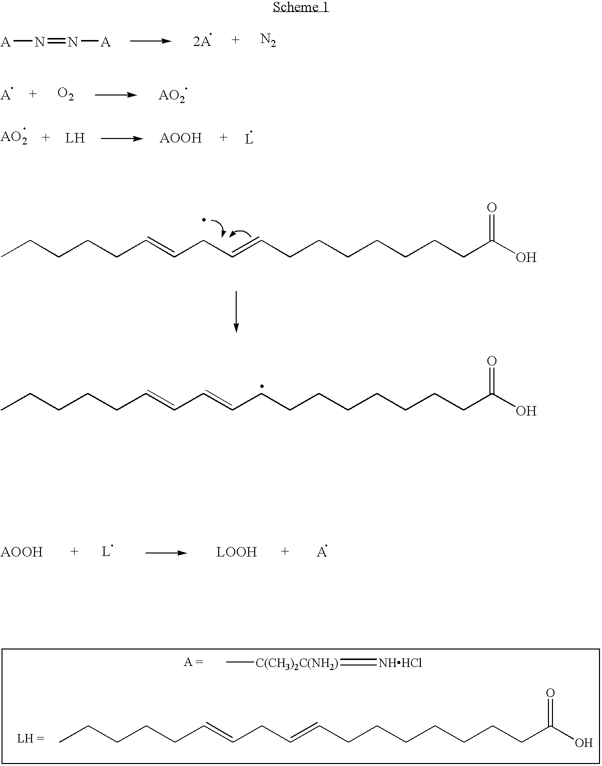

The efficiency is determined according to a standard, that is the generation of free radicals in the absence of an antioxidant. Efficiency is then calculated using the following equation:
Efficiency (%)=1−[K₂/K₁]×100
Where:
K₁=rate of oxidation of standard (no antioxidant)=[difference in absorbance]/[time (sec)]
K₂=rate of oxidation with antioxidant=[difference in absorbance]/[time (sec)]

The process described is a biomimetic process designed to generate some of the same free radicals as the body generates. Oxidation does not occur through the addition of hydrogen peroxide.

Prior to testing the efficiency of the compounds of the present invention, a number of well known antioxidants including vitamin E and ascorbic acid were tested. Each of the tests for vitamin E and ascorbic acid gave similar results to those presented in the literature. A number of other variables were tested including the amounts of antioxidant, substrate (linoleic acid) and initiator (AAPH). The optimal volumes are shown below:
2.78 ml 0.05M phosphate buffer pH 7.4
30 ul of linoleic acid dispersion stock
10 ul of 0.01M antioxidant stock

150 ul of 40 mM MPH stock

| Antioxidant | Concentration | Final Concentration | Efficiency |
|---|---|---|---|
| Vitamin E | 0.01 M | 33.3 μM | 93% |
| Ascorbic Acid | 0.01 M | 33.3 μM | 74% |
| (3)* | 0.01 M | 33.3 μM | 20% |
| (4)* | 0.01 M | 33.3 μM | 83% |
| (5)* | 0.01 M | 33.3 μM | 47% |
| (6)* | 0.01 M | 33.3 μM | 43% |

*Numbering corresponds to numbered structures used in the synthetic schemes provided herein

When testing the efficiency of compounds of the present invention in the presence and absence of Ca²⁺, HEPES buffered saline was used instead of the standard phosphate buffer and CaCl₂ was used as a source of calcium ions.

| | | HBS without | HBS with | |
|---|---|---|---|---|
| | Solvents (%) | calcium (%) | calcium (%) | Efficiency |
| Vitamin E | Methanol (33%) | 67% | — | 90% |
| (4)* | DMSO(33%) | 67% | — | 93% |
| (6)* | DMSO(33%) | 67% | — | 46% |
| Vitamin E | Methanol (33%) | — | 67% | 94% |
| (4)* | DMSO(33%) | — | 67% | 93% |
| (6)* | DMSO(33%) | — | 67% | 35% |

*Numbering corresponds to numbered structures used in the synthetic schemes provided herein

### EXAMPLE 5

In Vitro Studies on Neuronal Survival

Cellular viability of NSC34 cells (neuroblastoma×spinal cord cell lines) was determined at 24 hours after injury by the reduction of 3-(4,5-dimethylthiazole-2-yl)-2,5-diphenyltetrazolium bromide (MTT) (see Cheung N S, et al. (1998) Neuropharmacology 37, 1419-1429), a measure of mitochondrial function, which is compromised in injured cells (see Kroemer et al., (1998) Ann Rev Physiol 60, 619-642). MTT was incubated with the neurones for 30 min at 37° C. and the reduced formazan product was lysed from the cells in 20% sodium dodecyl sulphate and 40% dimethylformamide, and the absorbance read at 590 nm (Ceres UV900C microplate reader; Biotek Instruments, USA). Cultures treated with an excess of hydrogen peroxide or Triton X-100 were taken as 100% cell death and the results were expressed as percentage vehicle control.

### EXAMPLE 6

In Vitro Studies on Neuronal Survival

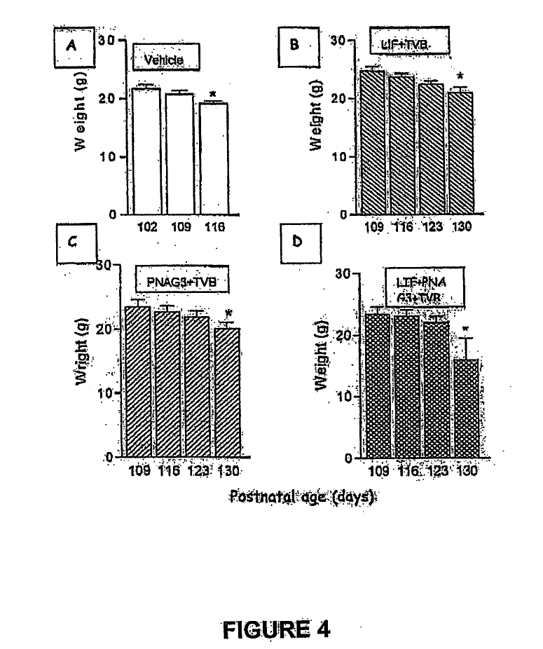

We developed two cell culture systems that can be used to screen drugs that prevent cell death induced by oxidant stress (FIG. 3, Model A) and excitotoxicity (FIG. 4, Model B). In model A, we utilize cultured fibroblasts that are exposed to paraquat that induces significant death at 10 μM (see FIG. 3). In model B we utilize the NSC-34 motor neuron cell line that can be induced to degenerate via the glutamate excitotixic pathway using the specific agonist fluorowillardine or FW (see FIG. 4).

### EXAMPLE 7

In Vivo Studies on Neuronal Survival

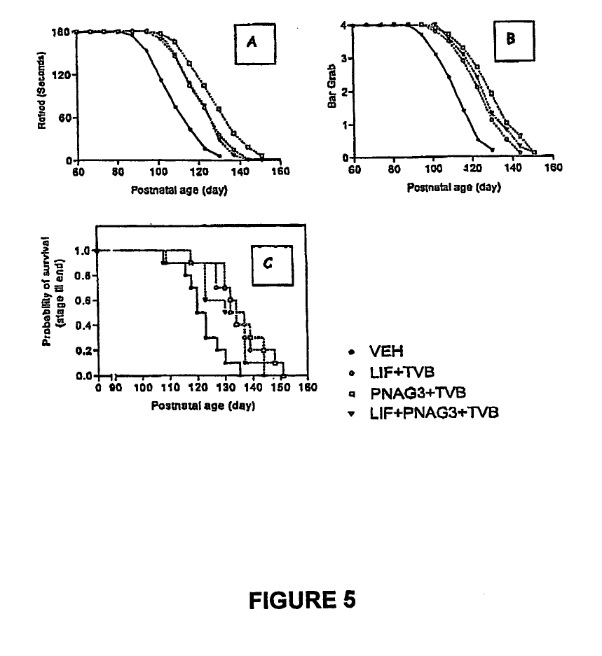

The tolerance and efficacy of compounds when they are administered as co-therapy with a peptide nucleic acid (herein referred to as PNAG3 and having the structure N-TCC GTG AGA ATG-C or N-GTG AGA ATG-C) and leukemia inhibitory factor (LIF) was determined. These experiments were carried out to determine if co-treatment with compound (3) has synergistic effects via the anti-oxidative and calcium buffering actions of compound (3). SOD1G93A mice tolerated VEH, PNAG3+compound (3), LIF+compound (3) and PNAG3+LIF+compound (3) therapy without significant adverse effects on behaviour and loss of body weight (FIG. 5). In all groups, the expected decline in weight due to the disease-associated atrophy of muscles was observed. Analysis of locomotor behaviour using the Rotarod apparatus (FIG. 6A) and bar grab task (FIG. 6B) shows that compared to the VEH group, the PNAG3+compound (3), LIF+compound (3) and PNAG3+LIF+compound (3) groups showed significant improvement. The survival of these mice is represented in FIG. 6C and Table 1. These data show that there is an improvement in survival.

TABLE 1
Tabular representation of onset of motor deficits on the Rotarod
test and the mean survival of the mice with MND. There is a statistical
difference in all three treated groups in Rotarod test (A) and average
survival days (B) compared to the vehicle group. In the tables, VEH =
vehicle, (3) is compound (3) as in the synthetic schemes presented
herein, LIF = leukaemia inhibitory factor and PNAG3 is N-TCC
GTG AGA ATG-C or N-GTG AGA ATG-C.

| | |
|---|---|
| Mean ± SD | ρ value |

(A) The onset of motor deficit on the Rotarod test

| | | |
|---|---|---|
| VEH | 101.3 ± 6.96 | |
| LIF + (3) | 114.6 ± 9.79 | <0.05 |
| PNAG3 + (3) | 120.2 ± 11.04 | <0.001 |
| LIF + PNAG3 + (3) | 118.8 ± 8.22 | <0.001 |

(B) Average survival days

| | | |
|---|---|---|
| VEH | 122 ± 7.57 | |
| LIF + (3) | 136 ± 9.79 | <0.05 |
| PNAG3 + (3) | 133 ± 10.33 | <0.05 |
| LIF + PNAG3 + (3) | 132 ± 10.01 | <0.05 |

Finally, it will be appreciated that various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are apparent to those skilled in the art are intended to be within the scope of the present invention.

1-49. (canceled)

50. A method for preventing or reducing the effects of oxidative stress on a substrate, the method including the step of preventing or decreasing the formation of reactive oxygen species in the substrate by treating the substrate with a compound of formula (I), or a pharmaceutically acceptable salt thereof: 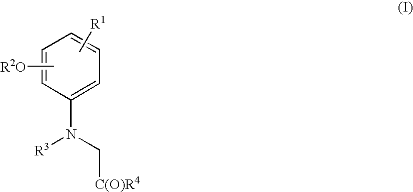
wherein:
R¹ is one or more substituents selected from —H, -alkyl, -alkoxy, -aryl, -aryloxy, -halogen, -amino (mono-, di- and tri-substituted), -alkylthio, —NO₂, —COOH, —COOAlkyl, —CO-alkyl, —CN;
R² is one or more substituents selected from —H, -alkyl, —(CH₂CH₂O)ₙ—R⁵ (n=1-5), a sugar moiety, -alkylbenzopyran (optionally substituted);
R³ is —H, -alkyl, -aryl, -alkylOR⁶, -alkylC(O)R⁶;
R⁵ is selected from —H, -alkyl, -aryl, the aryl ring shown in formula (I); and
R⁴ and R⁶ are independently selected from —OH, —O-alkyl, —O-polyalkyleneoxy, —O-aryl, —OC(O)O-alkyl, —S-alkyl and -amino.

51. A method as in claim 50 wherein R⁵ has the following formula: 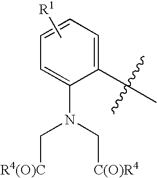
wherein:
R¹ is one or more substituents selected from —H, -alkyl, -alkoxy, -aryl, -aryloxy, -halogen, -amino, -alkylthio, —NO₂, —COOH, —COOAlkyl, —CO-alkyl, —CN; and
R⁴ is selected from —OH, —O-alkyl, —O-polyalkyleneoxy, —O-aryl, —OC(O)O-alkyl, —S-alkyl and -amino.

52. A method as in claim 51 wherein in the compound of formula (I) R² is —CH₂CH₂O—R⁵, R³ is —(CH₂C(O)R⁶, R⁴ and R⁶ are one or more of —O-methyl, —O-ethyl, —OCH₂CH₂OCH₂CH₂OCH₂CH₂OCH₃ or —OC(O)CH₃.

53. A method as in claim 50 wherein the compound of formula (I) has the following formula:

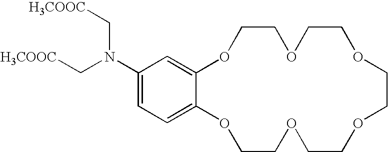

54. A method as in claim 50 wherein the compound of formula (I) has the following formula:

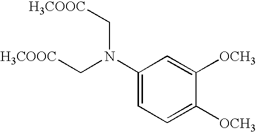

55. A method as in claim 50 wherein the compound of formula (I) has the following formula:

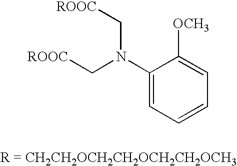

56. A method as in claim 51 wherein the compound of formula (I) has the following formula:

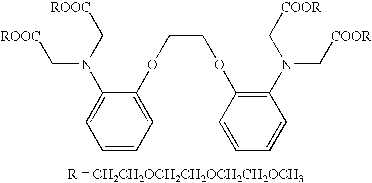

57. A method as in claim 50 wherein the compound of formula (I) has the following formula: 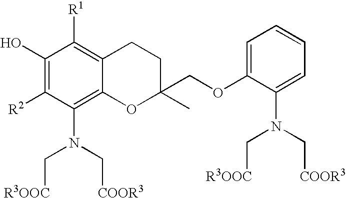
wherein
R¹ and R² are independently selected from —H, -alkyl, -alkoxy, -aryl, -aryloxy, -halogen, -amino, -alkylthio, —NO₂, —COOH, —COOAlkyl, —COAlkyl, —CN; and
R³ is selected from —OH, —Oalkyl, —OAryl, —Salkyl, amino, a sugar moiety, -polyalkyleneoxy, and a water solubilising group.

58. A method as in claim 50 wherein the substrate is a biological system.

59. A method as in claim 58 wherein the method is used to prevent or cure a neurodegenerative disorder.

60. A method as in claim 59 wherein the neurodegenerative disorder is stroke, epilepsy, Parkinson's disease, Huntington's disease, amyotrophic lateral sclerosis, multiple sclerosis or Alzheimer's disease.

61. A method as in claim 60 wherein treatment of the neurodegenerative disorder also involves administration of a second agent for treating a neurodegenerative disorder.

62. A method as in claim 61 wherein the second agent is a peptide nucleic acid.

63. A method as in claim 62 wherein the peptide nucleic acid is N-TCC GTG AGA ATG-C [SEQ ID NO:______] or N-GTG AGA ATG-C [SEQ ID NO:______].

64. A method as in claim 61 wherein the second agent is a neurotrophic factor.

65. A method as in claim 64 wherein the neurotrophic factor is leukaemia inhibitory factor.

66. A method of treating a disease state that is associated with calcium toxicity and oxidative stress, the method including the step of administering a therapeutically effective amount of a free radical scavenger and a calcium buffer.

67. A method as in claim 66 wherein the disease state that is associated with calcium toxicity and oxidative stress is a neurodegenerative disorder.

68. A method as in claim 18 wherein the neurodegenerative disorder is stroke, epilepsy, Parkinson's disease, Huntington's disease, amyotrophic lateral sclerosis, multiple sclerosis, and Alzheimer's disease.

69. A method as in claim 68 wherein the method includes the steps of administering a first agent that is free radical scavenger and a second agent that is a calcium buffer.

70. A method as in claim 69 wherein the free radical scavenger is lipoic acid, a 2,3-dihydro-1-benzofuran-5-ol, a chromanone, trolox or vitamin E.

71. A method as in claim 70 wherein the calcium buffer is 15-crown-5, 18-crown-6 or bis(o-aminophenoxy)ethane-N,N′,N′-tetraacetic acid.

72. A method as in claim 69 wherein the first and second agents are the same compound that is both a free radical scavenger and a calcium buffer.

73. A method as in claim 72 wherein the single agent is a compound of formula (I): 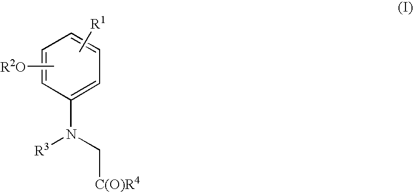
wherein:
R¹ is one or more substituents selected from —H, -alkyl, -alkoxy, -aryl, -aryloxy, -halogen, -amino (mono-, di- and tri-substituted), -alkylthio, —NO₂, —COOH, —COOAlkyl, —CO-alkyl, —CN;
R² is one or more substituents selected from —H, -alkyl, —(CH₂CH₂O)ₙ—R⁵ (n=1-5), a sugar moiety, -alkylbenzopyran (optionally substituted);
R³ is —H, -alkyl, -aryl, -alkylOR⁶, -alkylC(O)R⁶;
R⁵ is selected from —H, -alkyl, -aryl, the aryl ring shown in formula (I); and
R⁴ and R⁶ are independently selected from —OH, —O-alkyl, —O-polyalkyleneoxy, —O-aryl, —OC(O)O-alkyl, —S-alkyl and -amino.

74. A method as in claim 73 wherein R⁵ has the following formula: 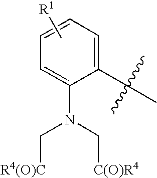
and wherein:
R¹ is one or more substituents selected from —H, -alkyl, -alkoxy, -aryl, -aryloxy, -halogen, -amino, -alkylthio, —NO₂, —COOH, —COOAlkyl, —CO-alkyl, —CN; and
R⁴ is selected from —OH, —O-alkyl, —O-polyalkyleneoxy, —O-aryl, —OC(O)O-alkyl, —S-alkyl and -amino.

75. A method as in claim 74 wherein in the compound of formula (I) R is —CH₂CH₂O—R⁵, R³ is —CH₂C(O)R⁶, R⁴ and R⁶ are one or more of —O-methyl, —O-ethyl, —OCH₂CH₂OCH₂CH₂OCH₂CH₂OCH₃ or —OC(O)CH₃.

76. A method as in claim 73 wherein the compound of formula (I) has the following formula:

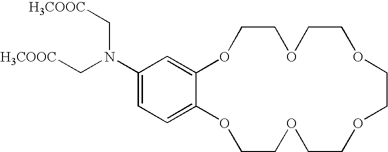

77. A method as in claim 73 wherein the compound of formula (I) has the following formula:

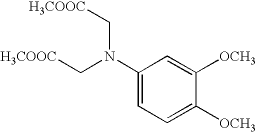

78. A method as in claim 73 wherein the compound of formula (I) has the following formula:

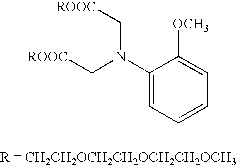

79. A method as in claim 74 wherein the compound of formula (I) has the following formula:

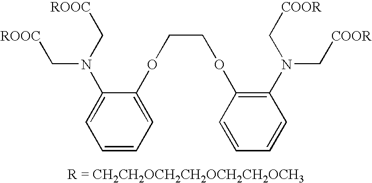

80. A method as in claim 73 wherein the compound of formula (I) has the following formula: 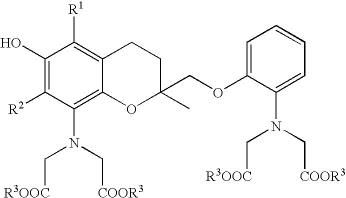
wherein
R¹ and R² are independently selected from —H, -alkyl, -alkoxy, -aryl, -aryloxy, -halogen, -amino, -alkylthio, —NO₂, —COOH, —COOAlkyl, —COAlkyl, —CN; and
R³ is selected from —OH, —Oalkyl, —OAryl, —Salkyl, amino, a sugar moiety, -polyalkyleneoxy, and a water solubilising group.

81. A compound of formula (II), or a pharmaceutically acceptable salt thereof: 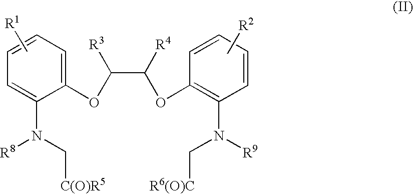
wherein:
R¹ and R² are each independently selected from one or more substituents selected from —H, -alkyl, -alkoxy, -aryl, -aryloxy, -halogen, -amino, -alkylthio, —NO₂, —COOH, —COOAlkyl, —CO-alkyl, —CN;
R¹ and R² are independently tetra-, tri- di- or mono-substitutions on each aromatic ring;
R³ and R⁴ are each independently selected from —H, -alkyl, —CH₂OH, -aryl, a sugar moiety, -polyalkyleneoxy, a water solubilising group, an antioxidant;
R⁵ and R⁶ are independently selected from —O-alkyl (C3 to C10), —O-aryl, —S-alkyl and -amino;
R⁸ and R⁹ are each independently selected from —H, -alkyl, —COOH, —COOAlkyl.

82. A compound as in claim 81 wherein the compound of formula (II) is in the form of a metal salt or an alkali earth metal complex.

83. A method for preventing or reducing the effects of oxidative stress on substrate, the method including the step of preventing or decreasing the formation of reactive oxygen species in the substrate by treating the substrate with a compound of formula (II), or a pharmaceutically acceptable salt thereof: 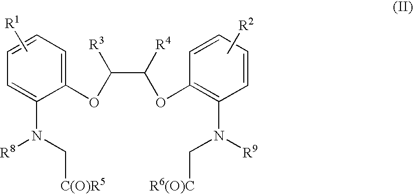
wherein:
R¹ and R² are each independently selected from one or more substituents selected from —H, -alkyl, -alkoxy, -aryl, -aryloxy, -halogen, -amino, -alkylthio, —NO₂, —COOH, —COOAlkyl, —CO-alkyl, —CN;
R¹ and R² are independently tetra-, tri- di- or mono-substitutions on each aromatic ring;
R³ and R⁴ are each independently selected from —H, -alkyl, —CH₂OH, -aryl, a sugar moiety, -polyalkyleneoxy, a water solubilising group, an antioxidant;
R⁵ and R⁶ are independently selected from —O-alkyl (C3 to C10), —O-aryl, —S-alkyl and -amino;
R⁸ and R⁹ are each independently selected from —H, -alkyl, —COOH, —COOAlkyl.

84. A method as claim 83 wherein the substrate is a biological system.

85. A method as in claim 84 wherein the method is used to prevent or cure a neurodegenerative disorder.

86. A method as in claim 85 wherein the neurodegenerative disorder is stroke, epilepsy, Parkinson's disease, Huntington's disease, amyotrophic lateral sclerosis, multiple sclerosis or Alzheimer's disease.

87. A formulation for scavenging free radicals, the formulation containing an effective amount of a compound of formula (I) or (II): 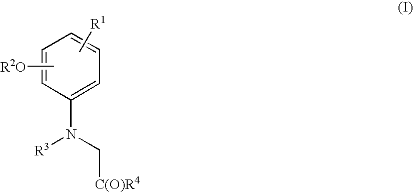
wherein:
R¹ is one or more substituents selected from —H, -alkyl, -alkoxy, -aryl, -aryloxy, -halogen, -amino (mono-, di- and tri-substituted), -alkylthio, —NO₂, —COOH, —COOAlkyl, —CO-alkyl, —CN;
R² is one or more substituents selected from —H, -alkyl, —(CH₂CH₂O)ₙ—R⁵ (n=1-5), a sugar moiety, -alkylbenzopyran (optionally substituted);
R³ is —H, -alkyl, -aryl, -alkylOR⁶, -alkylC(O)R⁶;
R⁵ is selected from —H, -alkyl, -aryl, the aryl ring shown in formula (I); and
R⁴ and R⁶ are independently selected from —OH, —O-alkyl, —O-polyalkyleneoxy, —O-aryl, —OC(O)O-alkyl, —S-alkyl and -amino, and 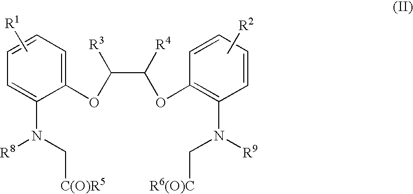
wherein:
R¹ and R² are each independently selected from one or more substituents selected from —H, -alkyl, -alkoxy, -aryl, -aryloxy, -halogen, -amino, -alkylthio, —NO₂, —COOH, —COOAlkyl, —CO-alkyl, —CN;
R¹ and R² are independently tetra-, tri- di- or mono-substitutions on each aromatic ring;
R³ and R⁴ are each independently selected from —H, -alkyl, —CH₂OH, -aryl, a sugar moiety, -polyalkyleneoxy, a water solubilising group, an antioxidant;
R⁵ and R⁶ are independently selected from —O-alkyl (C3 to C10), —O-aryl, —S-alkyl and -amino;
R⁸ and R⁹ are each independently selected from —H, -alkyl, —COOH, —COOAlkyl.
